(19) United States
(12) Patent Application Publication
(10) Pub. No.: US 2007/0270590 A1
(43) Pub. Date: Nov. 22, 2007

(54) METHODS FOR PREPARING ESZOPICLONE CRYSTALLINE FORM A, SUBSTANTIALLY PURE ESZOPICLONE AND OPTICALLY ENRICHED ESZOPICLONE

(76) Inventors: Marioara MENDELOVICI, Rehovot (IL); Anita LIBERMAN, Tel-Aviv (IL); Alex MAINFELD, Kfar-Saba (IL); Nina FINKELSTEIN, Herzliya (IL)

Correspondence Address: KENYON & KENYON LLP, ONE BROADWAY, NEW YORK, NY, 10004, US

(21) Appl. No.: 11/738,115

(22) Filed: Apr. 20, 2007

Related U.S. Application Data
(60) Provisional application No. 60/793,303, filed on Apr. 20, 2006.
(60) Provisional application No. 60/884,109, filed on Jan. 9, 2007.

(51) Int. Cl. C07D 487/02

(52) U.S. Cl. 544/350

(57) ABSTRACT

The present invention provides methods for preparing eszopiclone Form A, substantially chemically pure eszopiclone, or eszopiclone with low level(s) of residual solvent(s). The present invention also provides eszopiclone with low level(s) of residual solvent(s). The present invention also provides a process for optical enrichment of eszopiclone free base. For instance, one of the embodiments of the invention is directed to a method of preparing eszopiclone Form A, wherein the method comprises crystallizing eszopiclone free base from a solvent selected from the group consisting of isopropanol (IPA), methyl isobutyl ketone (MIBK), acetone, n-butanol, i-butanolisobutanol, 2-butanol, tetrahydrofuran (THF), dimethyl carbonate, methanol, ethanol, ethyl lactate, dimethylformamide (DMF), carbon tetrachloride, toluene, iso-butyl acetate and mixtures thereof.

### CROSS REFERENCE TO RELATED APPLICATIONS

The present application claims the benefits of U.S. Provisional Application Nos. 60/793,303 filed Apr. 20, 2006 and 60/884,109 filed Jan. 9, 2007, the disclosures of which are incorporated by reference.

### FIELD OF THE INVENTION

The present invention relates to methods for preparing eszopiclone crystalline Form A, substantially pure eszopiclone, optically enriched eszopiclone and eszopiclone with a low concentration of residual solvent(s).

### BACKGROUND

Zopiclone, a non-benzodiazepine which can be used to induce a sedative, hypnotic or tranquilizing effect, useful for treating insomnia, is a racemate having a chemical name of 4-methyl-1-piperazinecarboxylic acid 6-(5-chloro-2-pyridinyl)-6,7-dihydro-7-oxo-5H-pyrrolo[3,4-b]pyrazin-5-yl ester, (±)-6-(5-chloro-2-pyridinyl)-6,7-dihydro-7-oxo-5H-pyrrolo[3,4-b]pyrazin-5-yl-4-methylpiperazine-1-carboxylate, or 6-(5-chloropyrid-2-yl)-5-(4-methylpiperazin-1-yl)carbonyloxy-7-oxo-6,7-dihydro-5H-pyrrolo[3,4-b]pyrazine, represented with formula I below.

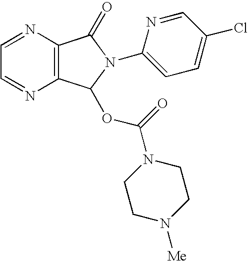

Eszopiclone is the S-enantiomer of zopiclone and is more active and less toxic than the racemic zopiclone according to U.S. Pat. No. 6,444,673 B1. This drug has been marketed in the United States by Sepracor™ under the name Lunesta®, formerly known as Estorra®, having a CAS Registry Number of 138729-47-2. Eszopiclone has a chemical name of (+)-6-(5-chloro-2-pyridinyl)-7(S)-(4-methylpiperazin-1-yl-carbonyloxy)-6,7-dihydro-5H-pyrrolo[3,4-b]pyrazine-5-one and is represented with formula II below.

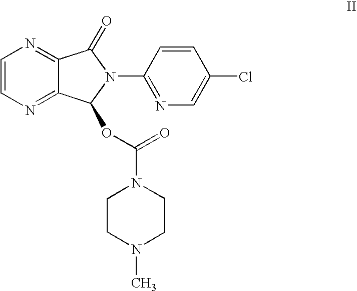

Eszopiclone in free base form and salt forms are disclosed in U.S. Pat. Nos. 6,444,673 and 6,864,257.

Eszopiclone can be prepared by optical resolution of racemic zopiclone. Blaschke, G. et al., Chirality (1993) 5:419-421 discloses preparation of eszopiclone free of its enantiomer using 0.5 equivalent D-(+)-Malic acid. By this procedure the diastereomeric salt is crystallized from a mixture of methanol-acetone, the salt is then neutralized and the free base is extracted in CH₂Cl₂/ethyl acetate and precipitated by concentration of the solution. The preparation of eszopiclone by optical resolution of racemic zopiclone using malic acid was improved in U.S. Pat. No. 6,339,086 using one equivalent D-(+)-Malic acid. In another method for optical resolution of racemic zopiclone, a semi-preparative HPLC method is described in C. F. F. Gimenez et. al., Chirality (1995) 7:267-271. A capillary electrophoresis method for zopiclone enantiomer separation is disclosed in High, J. Resolution Chromatography (2000) 23(6):413-429. Alternatively, US 2005/0043311A1 discloses eszopiclone separated from the racemic zopiclone using D-(+)-O,O′-dibenzoyl-tartaric acid. According to this method, eszopiclone is crystallized from acetonitrile.

These methods described afford eszopiclone in a free base form or salt forms, containing the unwanted enantiomer. Thus, there is a need in the art for improved processes for improving the optical purity of eszopiclone in diastereomeric salts or free base form.

Zopiclone exits in a few crystalline forms; these forms are characterized in Chem. Commun. (2001) 2204-2205 and J. Phys. IV France 11 (2001) pr10-93-pr10-97, and mentioned in Drug Development and Industrial Pharmacy (2000) 26(5):531-537. The crystalline forms are monoclinic dihydrate Form I, monoclinic anhydrous Form II and orthorhombic Form III.

Eszopiclone Form A, which has an X-ray diffraction (XRD) pattern equivalent to that of Form III of Zopiclone disclosed in the Chem. Commun. publication, is characterized by the following main XRD peaks: 5.1, 10.1, 11.3, 12.6, 16.1, 18.1, 19.1, 20.2, 21.4, 25.7, 27.7 ±0.2 degrees 2 theta. Form III is a racemic conglomerate suggesting that the same X-ray diffraction (XRD) pattern is exhibited also by a single enantiomer. It has been found that crystallization of eszopiclone free base in acetonitrile or ethylacetate in accordance with the disclosures of U.S. Pat. No. 6,444,673 and U.S. Pat. No. 6,339,086 produces polymorphic Form A.

However, acetonitrile is expensive and the solubility of eszopiclone in ethylacetate is low and a large volume of the solvent is required to induce complete dissolution. In addition, in order to recover eszopiclone from the solution in ethylacaetate concentration is required in order to induce eszopiclone precipitation. There is a need in the art for a method allowing industrial preparation of eszopiclone Form A using more practical solvents.

### SUMMARY OF THE INVENTION

The present invention provides a method for preparing eszopiclone Form A comprising crystallizing eszopiclone free base from a solvent selected from the group consisting of isopropanol (IPA), methyl isobutyl ketone (MIBK), acetone, n-butanol, isobutanol, 2-butanol, tetrahydrofuran (THF), dimethyl carbonate, methanol, ethanol, ethyl lactate, dimethylformamide (DMF), carbon tetrachloride, toluene, isobutyl acetate and mixtures thereof.

The present invention provides a method of preparing substantially chemically pure eszopiclone, comprising crystallizing eszopiclone free base from a solvent selected from the group consisting of methyl isobutyl ketone, isobutyl acetate, acetone, isobutanol, isopropanol (IPA), THF, toluene, ethanol:water (preferably in a volume ratio of about 1:1), n-butanol, 2-butanol and isopropanol:water (preferably in a volume ratio ranging from about 3:1 to about 10:1, e.g., about 3:1, about 5:1, about 7:1 or about 10:1).

The present invention is further directed to a method for optical enrichment of eszopiclone free base comprising crystallizing eszopiclone free base from a solvent selected from the group consisting of n-butanol, 2-butanol, isobutyl acetate, isobutanol, isopropanol (IPA), toluene, acetone, isopropanol:water (preferably in a volume ratio of about 3:1 to about 10:1), ethanol:water (preferably in a volume ratio of about 1:1 to about 1:27) and acetone:water (preferably in a volume ratio of about 1:1).

Another embodiment of the present invention provides eszopiclone free base with low level(s) of residual solvent(s).

Another embodiment of the present invention provides a method for preparing eszopiclone free base with low level(s) of residual solvent(s), comprising crystallizing eszopiclone from a solvent selected from the group consisting of: toluene, isobutyl acetate, isopropanol:water in a volume ratio of about 3:1 to about 10:1, and ethanol:water in a volume ratio of about 1:1 to about 95:5.

### DETAILED DESCRIPTION OF THE INVENTION

As used herein, “eszopiclone” refers to eszopiclone free base.

As used herein, “eszopiclone Form A” is crystalline eszopiclone characterized by the following main XRD peaks: 5.1, 10.1, 11.3, 12.6, 16.1, 18.1, 19.1, 20.2, 21.4, 25.7, 27.7 ±0.2 degrees 2 theta.

As used herein, “ambient temperature” refers to room temperature and is meant to indicate a temperature of about 18 to about 25° C., e.g., about 20 to about 22° C.

As used herein, “substantially chemically pure” refers to more than about 97%, e.g., at least about 99%, chemical purity as measured by HPLC.

As used herein, “substantially optically pure” in reference to eszopiclone means having at least about 97% of the (S)-enantiomer of zopiclone, as measured by chiral HPLC.

As used herein, eszopiclone free base with “low level(s) of residual solvent(s)” means that the eszopiclone contains residual solvent(s) in a content of no more than about 5000 ppm of isobutyl acetate, isopropanol or ethanol, and/or no more than about 1400 ppm of toluene, by weight of the eszopiclone product, wherein the residual solvent(s) is the solvent(s) used to crystallize the eszopiclone that remains associated with the eszopiclone product after crystallization.

As used herein, the solution of eszopiclone free base may be prepared, for example, by heating to a temperature of about 50° C. to about reflux. In addition, admixing additional solvent can aid in dissolution. The volume of solvent used may be determined based on the solubility of eszopiclone in each specific solvent.

The present invention provides a method for preparing eszopiclone Form A comprising crystallizing eszopiclone free base from a solvent selected from the group consisting of isopropanol (IPA), methyl isobutyl ketone (MIBK), acetone, n-butanol, isobutanol, 2-butanol, tetrahydrofuran (THF), dimethyl carbonate, methanol, ethanol, ethyl lactate, dimethylformamide (DMF), carbon tetrachloride, toluene, isobutyl acetate and mixtures thereof. Alternatively, eszopiclone free base may be slurried in a solvent selected from the group described above.

Stirring during crystallization of the eszopiclone Form A is preferred.

The precipitating step may be done by either cooling the solution or the slurry, or adding an anti-solvent to the solution or slurry.

Preferably, prior to the precipitation of the eszopiclone Form A, heating is performed. Preferably the heating is to a temperature ranging from ambient temperature to about reflux temperature.

Typically, the cooling is to a temperature ranging from about −10° C. to about ambient temperature.

The anti-solvent is preferably a non-polar solvent like ether, aromatic hydrocarbon and aliphatic hydrocarbon. More preferably, the anti-solvent is selected from the group consisting of: n-hexane, n-heptane, methyl tert-butyl ether (MTBE) and water. The volume of the anti-solvent added to a solution of eszopiclone can range from about 1 to about 50 volumes of the solution of eszopiclone.

The obtained crystalline form of eszopiclone Form A may be further recovered. Recovery of eszopiclone Form A may be by any means known to a skilled artisan such as by filtering, washing and drying, for example, in a vacuum oven.

Preferably, the obtained eszopiclone Form A is substantially chemically pure.

Preferably, the obtained eszopiclone Form A is substantially optically pure.

The present invention provides a method of preparing substantially chemically pure eszopiclone, comprising crystallizing eszopiclone free base from a solvent selected from the group consisting of methyl isobutyl ketone, isobutyl acetate, acetone, isobutanol, isopropanol (IPA), THF, toluene, ethanol:water (preferably in a volume ratio of about 1:1), n-butanol, 2-BuOH and isopropanol:water (preferably in a volume ratio ranging from about 3:1 to about 10:1, e.g., about: 3:1, 5:1, 7:1 or 10:1). Alternatively, eszopiclone free base may be slurried in a solvent selected from the group described above.

The process parameters are as described above. Preferably, the solvent is selected from the group consisting of MIBK, isopropyl alcohol, THF, toluene, ethanol/water in a volume ratio of about 1:1, acetone, 2-butanol, isopropyl alcohol/water in a volume ratio of about 3:1, about 5:1, about 7:1 or about 10:1, and isobutyl acetate.

The obtained eszopiclone is preferably eszopiclone Form A.

Preferably, the substantially chemically pure eszopiclone obtained by this process has a chemical purity of more than about 98%, more preferably more than about 99%, even more preferably more than about 99.5% and most preferably about 99.9% or higher, by HPLC.

The present invention is further directed to a method for optical enrichment of eszopiclone free base comprising crystallizing eszopiclone free base from a solvent selected from the group consisting of n-butanol, 2-butanol, isobutyl acetate, isobutanol, isopropanol (IPA), toluene, acetone, IPA/H₂O (preferably in a volume ratio of about 3:1 to about 10:1), ethanol:water (preferably in a volume ratio of about 1:1 to about 1:27) and acetone:water (preferably in a volume ratio of about 1:1). Alternatively, eszopiclone free base may be slurried in a solvent selected from the group described above.

The process parameters are as described above.

Preferably, the solvent is selected from the group consisting of toluene, isopropyl alcohol, n-butanol, isopropyl alcohol/water in a volume ratio of about 3:1, about 5:1, about 7:1 or about 10:1, and isobutyl acetate.

More preferably, the isopropyl alcohol to water volume ratio is of about 7:1 to 10:1.

Typically, the obtained substantially optically pure solid eszopiclone is eszopiclone Form A.

Preferably, the eszopiclone is optically enriched by 0.5%, more preferably optically enriched by 4% and most preferably optically enriched by 7%, compared with the starting eszopiclone. For instance, eszopiclone optically enriched by 4% means that the optical purity of the eszopiclone product is 4% higher than the optical purity of the starting eszopiclone as determined by chiral HPLC.

The obtained eszopiclone is substantially optically pure. The substantially optically pure eszopiclone obtained by this method can have an optical purity of preferably more than about 98%, more preferably more than about 99%, more preferably more than about 99.5% and most preferably, at least about 99.9%, by chiral HPLC.

When the mixture of isopropanol and water used has a volume ratio of about 5:1 or 10:1, the residual solvent(s) in the final eszopiclone product is no more than about 5000 ppm, preferably no more than about 800 ppm, and more preferably no more than about 700 ppm. This method is advantageous over the process disclosed in U.S. Pat. No. 6,339,086 in that the residual solvent content of the eszopiclone product is lower.

Preferably, the method comprises dissolving eszopiclone in a mixture of isopropanol and water; heating; and cooling to obtain a precipitate. Preferably, the heating is to a temperature of about ambient temperature to about reflux temperature, more preferably, to about reflux temperature. Preferably, the cooling is to a temperature ranging from about 0° C. to about room temperature, more preferably, to a temperature of about 10° C. Preferably, after cooling, a slurry is obtained. Preferably, the slurry is stirred. Preferably, the stirring is for about 30 minutes to about 20 hours.

Preferably, the obtained eszopiclone is eszopiclone Form A.

In some of the embodiments of the method, the obtained eszopiclone is optically enriched by at least about 0.1%, preferably optically enriched by at least about 4% and more preferably optically enriched by 4.5% compared with the starting eszopiclone as determined by chiral HPLC. For instance, the obtained eszopiclone can be optically enriched by about 0.1% or about 4%.

Preferably, the precipitate of the eszopiclone product is further recovered.

Recovery of eszopiclone, preferably Form A, in any of the above processes can be performed by any means known in the art for example by filtering, washing, and drying in vacuum. Preferably, the washing is with the same solvent used in the process. Preferably, when the solvent used in the process is IPA/water, the washing is with IPA. Preferably, the drying is at a temperature of about 30° C. to about 70° C., more preferably, at about 50° C. to about 60° C., and most preferably, at about 50° C.

Another embodiment of the present invention provides eszopiclone free base with low level(s) of residual solvent(s).

Another embodiment of the present invention provides a method for preparing eszopiclone free base with low level(s) of residual solvent(s), comprising crystallizing eszopiclone from a solvent selected from the group consisting of: toluene, isobutyl acetate, isopropanol:water in a volume ratio of about 3:1 to about 10:1, and ethanol:water in a volume ratio of about 1:1 to about 95:5. Alternatively, a mixture of these solvents can be used in the method.

The eszopiclone free base with low level(s) of residual solvent(s) preferably contains no more than about 1350 ppm toluene (more preferably no more than about 890 ppm toluene), no more than about 600 ppm isobutyl acetate or no more than about 800 ppm (more preferably no more than about 700 ppm) isopropanol.

Preferably, the process comprises providing a solution of eszopiclone free base in a solvent selected from the group consisting of: toluene, isobutyl acetate, isopropanol:water in a volume ratio of about 3:1 to about 10:1, and ethanol:water in a volume ratio of about 1:1 to about 95:5, and precipitating the eszopiclone. The eszopiclone can be precipitated from the solution by cooling or solvent removal via evaporation such as evaporation under vacuum.

Preferably, the process comprises combining eszopiclone free base with a solvent selected from the group consisting of: toluene, isobutyl acetate, isopropanol:water in a volume ratio of about 3:1 to about 10:1, and ethanol:water in a volume ratio of about 1:1 to about 95:5; heating; and cooling.

Preferably, the heating is to a temperature of about 30° C. to about 90° C., more preferably, to about 60° C. to about 80° C. Preferably, the cooling is to a temperature of less than about ambient temperature.

Preferably, the isopropanol:water volume ratio is of about 7:1 to 10:1

Preferably, the ethanol:water volume ratio is of about 3:1 to about 10:1, more preferably, of about 95:5.

Preferably, the obtained eszopiclone is substantially chemically pure.

Preferably, the obtained eszopiclone is substantially optically pure.

Preferably, the obtained eszopiclone is eszopiclone Form A.

The use of isopropanol:water and ethanol:water in a solution of eszopiclone with a limited volume of water is advantageous because it enables using a low amount of the organic solvent.

The identity of the eszopiclone Form A obtained by the processes of the invention is confirmed by analysis of the powder X-ray diffraction (XRD) pattern obtained by methods known in the art using a Scintag X-ray powder diffractometer having a variable goniometer, an X-Ray tube with Cu target anode (Cu radiation λ=1.5418 Å), a solid state detector and a round standard aluminum sample holder.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

HPLC method (with regard to the optical purity)

| | |
|---|---|
| Column & packing: | Chiralcel OD-H 4.6 × 250 mm, 5 μm CN 14325 |
| Eluent: | 0.1% DEA in Ethanol |
| Stop time: | 25 min |
| Flow: | 0.7 ml/min |
| Detector: | 306 nm |
| Injection volume: | 20 μl. |
| Diluent: | Ethanol |
| Column temperature: | 25° C. |
| Autosampler | 10° C. |
| temperature: | |
| Run time: | R-ZP: 12.9 min; S-ZP: 18.4 min |

HPLC method (with regard to the chemical purity)

| Column & packing: | Inertsil ODS 3 V 250*4.6 mm 5μ C.N | | |
|---|---|---|---|
| | 5020-01802 | | |
| Buffer preparation: | 0.01M Sodium dihydrogen phosphate | | |
| | adjusted to pH = 7.0 with 1N NaOH | | |
| Eluent A: | 66% Buffer:34% Acetonitrile | | |
| Eluent B: | Acetonitrile | | |
| Gradient of Eluent: | Time (min) | Eluent A (%) | Eluent B (%) |
| | 0 min | 100 | 0 |
| | 13 min | 100 | 0 |
| | 23 min | 40 | 60 |
| | 33 min | 40 | 60 |

| | |
|---|---|
| Stop time: | 33 min |
| Equilibration time: | 7 min |
| Flow: | 1.0 ml/min |
| Detector: | 306 nm. |
| Injection volume: | 20 μl. |
| Diluent: | 50% Acetonitrile:50% Buffer |
| Column temperature: | 25° C. |
| Autosampler temp.: | 5° C. |

### Example 1

### Preparation of Eszopiclone Form A from Ethyl Acetate [Comparative Example]

To eszopiclone free base (1 g) was added ethyl acetate (AR grade) (20 ml) and the slurry was heated to reflux. At reflux, an additional 10 ml of ethyl acetate were added to complete dissolution of the solid. Heating was stopped and the solution was cooled in ice-acetone bath to a temperature of about −10° C. in 15 min. Precipitation started at 60° C. The mixture was stirred at about −10° C. for 1 h. The solid was filtered, washed with 2 ml EtOAc and dried in a vacuum oven at 60° C. to yield eszopiclone Form A. Yield 80%.

### Example 2

### Preparation of Eszopiclone Form A from MIBK

To eszopiclone free base (1.5 gr) was added MIBK (CP grade) (6 ml) and the obtained slurry was heated to reflux. During heating an additional 6 ml of MIBK were added. At reflux additional solvent (9 ml) was added to induce complete dissolution of the solid. Heating was stopped and the solution was cooled to the room temperature. Precipitation started at 80° C. After this the mixture was cooled to 26° C. in 45 min. and stirred at room temperature for 2 h. The solid was filtered, washed with 2 ml MIBK and dried in vacuum oven at 65° C. The wet material was Eszopiclone Form A. Yield 80%. (Chemical Purity 99.96% by HPLC).

### Example 3

### Preparation of Eszopiclone Form A from N-Butanol

To eszopiclone free base (1.5 gr.) was added n-BuOH(CP grade) (6 ml) and the obtained slurry was heated to reflux. At reflux (114° C.), additional 4 ml of n-BuOH were added to slurry till full dissolution of the solid. Then heating was stopped and solution was cooled to the room temperature. Precipitation of solid started at 100° C. The mixture was cooled to 29° C. in about 40 min. and then stirred at room temperature (26° C.-29° C.) for 1 h. The solid was filtered, washed with 3 ml n-BuOH and dried in a vacuum oven at 65° C. and 7 mm Hg. Yield 90% (Chemical Purity 100% by HPLC).

### Example 4

### Preparation of Eszopiclone Form A from Isobutanol

To eszopiclone free base (1 gr) was added i-BuOH(CP grade) (6 ml) and the obtained slurry was heated to reflux. At reflux (106° C.), additional 4 ml of i-BuOH were added to slurry till full dissolution of the solid. Then heating was stopped and the solution was cooled to the room temperature. Precipitation of solid started at 97° C. The mixture was cooled to 24° C. in ˜35 min. and then stirred at room temperature (˜24° C.) for 2 h. The solid was filtered, washed with 2 ml i-BuOH and dried in a vacuum oven at 60° C. and 7 mm Hg. Yield 71% (Chemical Purity 97.37% by HPLC).

### Example 5

### Preparation of Eszopiclone Form A from Isopropanol

To eszopiclone free base (1.5 gr) was added IPA (HPLC grade) (3 ml) and the obtained slurry was heated to reflux. During heating additional 6 ml of IPA were added. At reflux (82° C.), additional 62 ml of IPA were added to slurry till full dissolution of the solid. Then heating was stopped and solution was cooled to room temperature. Precipitation of solid started at 60° C. The mixture was cooled to 25° C. in ˜40 min. and then stirred at room temperature (about 26° C.-29° C.) for 2 h. The solid was filtered, washed with 3 ml IPA and dried on a shelf over weekend. Then solid was dried at 60° C. Yield 80% (Chemical Purity 99.94% by HPLC).

### Example 6

### Preparation of Eszopiclone Form A from THF

To eszopiclone free base (1.5 gr) were added THF (extra dry) (7.5 ml) and slurry was heated to reflux. At reflux, additional 16 ml THF were added to full dissolution of solid. Heating was stopped and the solution was cooled to room temperature. Precipitation started at 45° C. The mixture was cooled to 27° C. in ˜1 h and then stirred at room temperature for 2 h. The solid was filtered, washed with 3 ml THF and dried in vacuum oven at 60° C. Yield 60% (Chemical Purity 99.93% by HPLC).

### Example 7

### Preparation of Eszopiclone Form A from Toluene

To eszopiclone free base (0.9 gr) was added toluene (extra dry) (4.5 ml) and slurry was heated to reflux. At reflux, additional toluene (4 ml) was added to complete dissolution of solid. Heating was stopped and solution was cooled to room temperature. Precipitation started at 70° C. The mixture was cooled to 26° C. in ˜50 min. and then stirred at room temperature for 2 h. The solid was filtered, washed with 1.5 ml toluene and dried in vacuum oven at 65° C. Yield 70% (Chemical Purity 99.72% by HPLC).

### Example 8

### Preparation of Eszopiclone Form A from EtOH/H₂O (1:1)

To eszopiclone free base (1.5 gr) was added a 1:1, based on volume, mixture of EtOH/H₂O (15 ml) and slurry was heated to reflux. At 76° C. complete dissolution of solid was observed. Heating was stopped at 80° C. and slurry was cooled to room temperature. Precipitation started at 68° C. The mixture was cooled to 28° C. in ˜50 min. and then stirred at room temperature for 2 h. The solid was filtered, washed with 3 ml solvents mixture and dried in a vacuum oven at 60° C. Yield 67% (Chemical Purity 99.65% by HPLC).

### Example 9

### Preparation of Eszopiclone Form A from I-BuOAc

To eszopiclone free base (1.5 gr) was added i-BuOAc (AR grade) (7.5 ml) and slurry was heated to reflux. At reflux, additional 18 ml of i-BuOAc were added to complete dissolution of solid. Heating was stopped and the solution was cooled to room temperature. Precipitation started at 85° C. The mixture was cooled to 28° C. in ˜50 min. and then stirred at room temperature for 2 h. The solid was filtered, washed with 3 ml i-BuOAc and dried in a vacuum oven at 50° C. Yield 93% (Chemical Purity by HPLC 99.95%).

### Table Summarizing Results of Example 2-9

| | | Solvent | | | |
|---|---|---|---|---|---|
| | | volume in | | | |
| | | ml per | | | |
| | | gram of | | | Chemical |
| | | starting | | | Purity |
| Example | Solvent | eszopiclone | Yield | XRD | by HPLC |
| 2 | MIBK | 14 | 80% | (wet) | 99.96% |
| | | | | Form A | |
| 3 | n-BuOH | 6.7 | 90% | (wet) | Not |
| | | | | Form A | analyzed |
| 4 | i-BuOH | 10 | 71% | (wet) | 97.3% |
| | | | | Form A | |
| 5 | IPA | 47 | 80% | (wet) | 99.94% |
| | | | | Form A | |
| 6 | THF | 15.7 | 60% | | 99.93% |
| 7 | toluene | 9.5 | 70% | Form A | 99.72% |
| 8 | EtOH/ | 10 | 67% | (wet) | 99.65% |
| | H₂O | | | Form A | |
| | (1:1) | | | | |
| 9 | i-BuOAc | 17 | 93% | (wet) | Not |
| | | | | Form A | analyzed |

### Examples 10-20

### Preparation of Eszopiclone Form A from Other Solvents by Method Described in Examples 2-9

Eszopiclone Form A was also prepared using other solvents shown in the table below with a method corresponding to that described in Examples 2-9.

| | | Solvent | | | |
|---|---|---|---|---|---|
| | | volume in | | | |
| | | ml per | | | |
| | | gram of | | | Chemical |
| | | starting | | | Purity |
| Example | Solvent | eszopiclone | Yield | XRD | by HPLC |
| 10 | acetone | 44 | 58% | (wet) | 100% |
| | | | | Form A | |
| 11 | EtOH/H₂O | 27/1 | 75% | (wet) | Not |
| | | | | Form A | analyzed |
| 12 | dimethyl | 10 | 60% | (wet) | Not |
| | carbonate | | | Form A | analyzed |
| 13 | MeOH | 10 | 54% | (wet) | Not |
| | | | | Form A | analyzed |
| 14 | Ethyl | 6 | 49% | | Not |
| | lactate | | | | analyzed |
| 15 | DMF | 5 | 63% | Form A | Not |
| | | | | | analyzed |
| 16 | CCl₄ | 78 | 53% | Form A | Not |
| | | | | | analyzed |
| 17 | Acetone/ | 70 | 51% | Form A | Not |
| | H₂O(1:1) | | | | analyzed |
| 18 | EtOAc/H₂O | 22.5 | 36% | Form A | Not |
| | (3% H₂O) | | | | analyzed |
| 19 | 2-BuOH | 28 | 87% | | 99.66% |
| 20 | EtOAc/H₂O | 30 | 56% | Form A | Not |
| | (1.5% H₂O) | | | | analyzed |

### Example 21

### Preparation of Eszopiclone Form A from THF as Solvent and MTBE as Anti-Solvent

To eszopiclone free base (0.8 gr) was added THF extra dry (12.5 ml) and the obtained slurry was heated to reflux. At reflux, the solid was dissolved to give a clear solution. To the solution was added MTBE (CP grade), until a solid started to precipitate. With MTBE addition, the temperature of the solution dropped. After 8 ml of MTBE were added, precipitation started at a temperature of about 60° C., heating was stopped and the slurry was cooled to room temperature. The mixture was cooled to 28° C. in 25 min and then stirred at 28° C. for 2 h. The solid was filtered, washed with 1.5 ml of MTBE and dried in a vacuum oven at 60° C. Yield 75%.

### Examples 22-26

### Preparation of Eszopiclone Form A from Other Solvent/Anti-Solvent by Method Described in Example 21

Eszopiclone Form A was also prepared using other solvents/antisolvents shown in the table below with a method corresponding to that described in Example 21.

| | | Volume Ratio | | |
|---|---|---|---|---|
| | | of Solvent/ | | |
| Example | Solvent/Antisolvent | Antisolvent | Yield | XRD |
| 22 | Toluene/n-Heptane | 9.5/1.7 | 73% | Form A |
| 23 | n-BuOH/MTBE | 11.1/7.2 | 87% | Form A |
| 24 | Acetone/MTBE | 40/55.5 | 57% | Form A |
| 25 | THF/MTBE | 15.6/10 | 75% | Form A |
| 26 | IPA/H₂O | 47/62.5 | 50% | Form A |

### Example 27-31

### Preparation of Optically Enriched Eszopiclone Form A by Method Described in Examples 2-9

| | | | Optical | Optical | |
|---|---|---|---|---|---|
| | | Solvent volume | purity of | purity of | |
| | | in ml per gram | starting | crystallized | |
| Exam- | | of starting | Eszopiclone | Eszopiclone | Yield |
| ple | Solvent | Eszopiclone | (by HPLC) | (by HPLC) | (%) |
| 27 | toluene | 9.5 | 92.5% | 99.9% | 78% |
| 28 | i-BuOAc | 17 | 92.5% | 96.66% | 88% |
| 29 | Isopropyl | 47 | 92.5% | 97.4% | 88% |
| | alcohol | | | | |
| 30 | isobutanol | 10 | 92.5% | 93.4% | 86% |
| 31 | n-Butanol | 10 | 92.5% | 97.2% | 90% |

### Examples 32-35

### Preparation of Eszopiclone Form A from IPA/Water

Eszopiclone (2.0 g, optical purity 95.5-98.5%) was dissolved in aqueous isopropanol (“IPA”) by heating at reflux. The solution was cooled with stirring to room temperature for an hour and stirred for additional hour at room temperature. The solid was filtered, washed with aqueous isopropanol, dried under vacuum at 50° C. overnight resulting in Eszopiclone crystalline form A with yield 85-95%.

| | Volume | | | | |
|---|---|---|---|---|---|
| | IPA/water | | | | |
| | (ml per | | | | |
| | gram of | Volume | HPLC purity % | | Residual |
| Exam- | starting | ratio | Chemical | Optical | solvent |
| ple | eszopiclone) | IPA:water | purity | purity | (ppm) |
| 32 | 39 | 3:1 | 99.96 | 99.91 | Not |
| | | | | | analyzed |
| 33 | 28 | 5:1 | 99.93 | 99.97 | 676 |
| 34 | 39 | 10:1 | 99.97 | 100 | 770 |
| 35 | 30 | 7:1 | 100 | 100 | Not |
| | | | | | analyzed |

### Example 36

### Preparation of Eszopiclone Form A from IPA/Water by Cooling to 10° C.

Eszopiclone (7 g, optical purity 98.45%) was dissolved in aqueous isopropanol 10:1 (136.5 ml) by heating at reflux. The solution was cooled with stirring to about 10° C. during one hour and stirred for additional two hours at that temperature. The solid was filtered, washed with aqueous iso-propanol, dried under vacuum at 50° C. overnight and gave Eszopiclone, having crystalline form A with yield 89.5%. (Optical purity: 99.91% by HPLC, Chemical purity: 99.4% by HPLC)

### Example 37

### Repetition of Example 2 from U.S. Pat. No. 6,339,086

To a mixture of Eszopiclone malate (2.0 g, 3.74 mmol, purity profile 99.96% by HPLC and optical purity 96% by HPLC) in water (4 ml) and ethyl acetate (20 ml), 40% aqueous potassium carbonate (1.6 g, 4.64 mmol) was added slowly with stirring at 30° C. Then the mixture was heated at 60° C., and the organic phase was isolated and washed with 20 ml of water. The mixture was concentrated to 2/3 volume of the organic solvent. The resulted slurry was cooled to 5° C. and stirred at the same temperature for additional 2 hours. The solid was filtered, washed with cold ethyl acetate, dried at 50° C. under vacuum overnight (the first drying) yielding Eszopiclone containing residual ethyl acetate at a level of 7534 ppm (GC) with an optical purity of 96.35% by HPLC. The Eszopiclone was further dried at 75° C. under vacuum for 18 hours (the second drying) yielding Eszopiclone containing residual ethyl acetate at a level of 7360 ppm (GC).

### Example 38

### Using Other Organic Solvents to Conduct a Process Similar to the Process in Example 2 of U.S. Pat. No. 6,339,086

To a mixture of Eszopiclone malate (2.0 g, 3.74 mmol, purity profile 99.96% by HPLC and optical purity 96%) in water (4 ml) and one of the organic solvents shown in the table below, 40% aqueous potassium carbonate (1.6 g, 4.64 mmol) was added slowly with stirring at 30° C. Then the mixture was heated, and the organic phase was isolated and washed with 20 ml of water. The mixture was concentrated. The resulted slurry was cooled to 5° C. and stirred at the same temperature for additional 2 hours. The solid was filtered, washed with cold organic solvent (as used before), dried at 50° C. under vacuum overnight (the first drying) to obtain Eszopiclone containing a residual solvent. The Eszopiclone was further dried at 75° C. under vacuum for 18 hours (the second drying) resulting in Eszopiclone containing a residual solvent.

The results of Examples 37 and 38 (involving preparation of eszopiclone from eszopiclone malate by neutralization, extraction with organic solvent and precipitation) are shown in the table below.

| | | Heating | | | | | | |
|---|---|---|---|---|---|---|---|---|
| | Volume of organic | temperature | | | | | Residual | Residual |
| | solvent in ml per | (during the | | | Chemical | Optical | solvent (ppm) | solvent (ppm) |
| Organic | gram of starting | extraction) | Yield | Recovery* | purity | purity | After 1^{st} | After 2^{nd} |
| Solvent | eszopiclone | (° C.) | (%) | (%) | (%) | (%) | drying | drying |
| Ethyl | 20 | 60 | 80 | | | 96.35 | 7534 | 7360 |
| acetate | | | | | | | | |
| Butyl | 54 | 80 | 73 | Not | Not | 96.4 | 6733 | 6468 |
| acetate | | | | checked | analyzed | | | |
| Toluene | 25 | 80 | 75 | Not | 99.95 | 96.8 | 8025 | 7478 |
| | | | | checked | | | | |
| isobutyl | 64 | 80 | 60 | 83 | 99.94** | 99.15 | 7799 | 7578 |
| acetate | | | | | | | | |

*The “Recovery” relates to the total amount of Eszopiclone (in its solid form and in the mother liquor)
**Material balance of Eszopiclone: Yield of the solid and the material in mother liquids in these experiments was less than 83%, while the purity profile of the material dissolved in mother liquids was about 95%, indicating decomposition of eszopiclone. In the water phase, eszopiclone was absent.

### Example 39

### Preparation of Eszopiclone from Eszopiclone Malate by Neutralization in Water Filtration and Crystallization from Organic Solvent

Example 39 was conducted using the procedure described in Example 38 using different solvents. The results of this experiment are shown in the table below.

Residual solvent in Eszopiclone prepared by crystallization

| Solvent, | | | | | |
|---|---|---|---|---|---|
| volume in relation to | | Chemical purity | Optical purity | Optical purity | Residual |
| starting Eszopiclone | Yield | of Eszopiclone | of starting | of Eszopiclone | solvent |
| (ml/gr) | (%) | product (%) | Eszopiclone (%) | product (%) | (ppm) |
| toluene (9) | 90 | 99.97 | 98.85 | 99.96 | 1349 |
| isobutyl acetate (27) | 80 | 99.95 | 98.85 | 99.97 | 568 |
| isopropanol/water, | 85 | 99.97 | 95.5 | 99.91 | 676 |
| volume ratio 5:1, (14) | | | | | |

1. A method for preparing eszopiclone Form A comprising crystallizing eszopiclone free base from a solvent selected from the group consisting of isopropanol, methyl isobutyl ketone, acetone, n-butanol, isobutanol, 2-butanol, tetrahydrofuran, dimethyl carbonate, methanol, ethanol, ethyl lactate, dimethylformamide, carbon tetrachloride, toluene, isobutyl acetate and mixtures thereof.

2. The method of claim 1, further comprising heating the eszopiclone free base in the solvent.

3. The method of claim 1, wherein the eszopiclone Form A is precipitated by cooling.

4. The method of claim 3, wherein the cooling is to a temperature between about −10° C. and ambient temperature.

5. The method of claim 1, wherein the solution of the eszopiclone free base in the solvent is stirred.

6. The method of claim 1, further comprising slurrying the eszopiclone free base in the solvent.

7. The method of claim 1, wherein the eszopiclone Form A is precipitated by the addition of at least one anti-solvent.

8. The method of claim 7, wherein the at least one anti-solvent is selected from the group consisting of non-polar organic solvents and water.

9. The method of claim 8, wherein the at least one anti-solvent is selected from the group consisting of ether, aromatic hydrocarbon, aliphatic hydrocarbon and water.

10. The method of claim 9, wherein the at least one anti-solvent is selected from the group consisting of methyl tert-butyl ether, n-hexane, n-heptane and water.

11. The method of claim 7, wherein the volume of the at least one anti-solvent ranges from about 1 volume to about 50 volumes based on the volume of the solution of eszopiclone free base.

12. The method of claim 7, further comprising slurrying the eszopiclone free base in the solvent and at least one anti-solvent.

13. The method of claim 1, wherein the solvent is selected from the group consisting of methyl isobutyl ketone, isopropyl alcohol, tetrahydrofuran, toluene, ethanol/water in a volume ratio of about 1:1, acetone, 2-butanol, isopropanol/water in a volume ratio of about 3:1, about 5:1, about 7:1 or about 10:1, and isobutyl acetate.

14. The method of claim 1, wherein the eszopiclone Form A product has a chemical purity of more than about 97% as measured by HPLC.

15. The method of claim 1, wherein the eszopiclone Form A product has an optical purity of at least about 97% as measured by HPLC.

16. A method of preparing eszopiclone having a chemical purity of more than about 97% as measured by HPLC, comprising crystallizing eszopiclone free base from a solvent selected from the group consisting of methyl isobutyl ketone, isobutyl acetate, acetone, isobutanol, isopropanol, tetrahydrofuran, toluene, ethanol:water, n-butanol, 2-butanol, isopropanol:water and mixtures thereof.

17. The method of claim 16, wherein the ethanol:water has a volume ratio of about 1:1, and the isopropanol:water has a volume ratio of about 3:1 to about 10:1.

18. The method of claim 16, wherein the solvent is selected from the group consisting of methyl isobutyl ketone, isopropanol, tetrahydrofuran, toluene, ethanol:water in a volume ratio of about 1:1, acetone, n-butanol, 2-butanol, isopropanol:water in a volume ratio of about 3:1, about 5:1, about 7:1 or about 10:1, and isobutyl acetate.

19. The method of claim 18, wherein the solvent is selected from the group consisting of ethanol:water in a volume ratio of about 1:1, methyl isobutyl ketone, isopropanol, tetrahydrofuran, acetone, isopropropanol:water in a volume ratio of about 3:1, about 5:1, about 7:1 or about 10:1, toluene and isobutyl acetate.

20. The method of claim 18, wherein the solvent is selected from the group consisting of methyl isobutyl ketone, isopropanol, tetrahydrofuran, acetone, isopropropanol:water in a volume ratio of about 3:1, about 5:1, about 7:1 or about 10:1, toluene and isobutyl acetate.

21. The method of claim 16, wherein the solution of the eszopiclone free base in the solvent is stirred.

22. The method of claim 16, further comprising slurrying the eszopiclone free base and the solvent.

23. The method of claim 16, wherein the eszopiclone product has a chemical purity of more than about 99%.

24. The method of claim 23, wherein the eszopiclone product has a chemical purity of more than about 99.5%.

25. The method of claim 24, wherein the eszopiclone product has a chemical purity of at least about 99.9%.

26. The method of claim 16, wherein the eszopiclone product is eszopiclone Form A.

27. A method for preparing eszopiclone free base having at least about 97% of the (S)-enantiomer of zopiclone as measured by HPLC, comprising crystallizing eszopiclone free base from a solvent selected from the group consisting of n-butanol, 2-butanol, isobutyl acetate, isobutanol, isopropanol, toluene, acetone, isopropanol:water, ethanol:water, acetone:water and mixtures thereof.

28. The method of claim 27, wherein the isopropanol:water mixture has a volume ratio of about 3:1 to about 10:1, the ethanol:water mixture has a volume ratio of about 1:1 to about 1:27, and the acetone:water mixture has a volume ratio of about 1:1.

29. The method of claim 28, wherein the solvent is selected from the group consisting of toluene, isopropanol:water in a volume ratio of about 3:1, about 5:1, about 7:1 or about 10:1, and isobutyl acetate.

30. The method of claim 28, wherein the solvent is an isopropanol:water mixture having a volume ratio of about 5:1 to about 10:1.

31. The method of claim 30, wherein the solvent is an isopropanol:water mixture having a volume ratio of about 7:1 to about 10:1.

32. The method of claim 27, wherein the solution of the starting eszopiclone free base in the solvent is stirred.

33. The method of claim 27, further comprising slurrying the starting eszopiclone free base and the solvent.

34. The method of claim 27, wherein the obtained eszopiclone free base product is eszopiclone Form A.

35. The method of claim 27, wherein the obtained eszopiclone free base product is optically enriched by at least about 0.1% compared with the starting eszopiclone free base.

36. The method of claim 35, wherein the obtained eszopiclone free base product is optically enriched by at least about 4% compared with the starting eszopiclone free base.

37. The method of claim 36, wherein the obtained eszopiclone free base product is optically enriched by about 7% compared with the starting eszopiclone free base.

38. The method of claim 27, wherein the obtained eszopiclone free base product has an optical purity of more than about 99%.

39. The method of claim 38, wherein the obtained eszopiclone free base product has an optical purity of more than about 99.5%.

40. The method of claim 39, wherein the obtained eszopiclone free base product has an optical purity of at least about 99.9%.

41. The method of claim 27, wherein the eszopiclone free base is dissolved in a mixture of isopropanol and water; followed by heating and then cooling to obtain a precipitate as the eszopiclone free base product having at least about 97% of eszopiclone.

42. The method of claim 41, wherein the heating is to about reflux temperature and the cooling is to a temperature ranging from about 0° C. to about room temperature.

43. The method of claim 41, wherein the cooling is to about 10° C.

44. The method of claim 41, wherein a slurry is obtained via cooling and the slurry is stirred.

45. The method of claim 44, wherein the slurry is stirred from about 30 minutes to about 20 hours.

46. Eszopiclone having no more than about 5000 ppm of isobutyl acetate, isopropanol or ethanol, and/or no more than about 1400 ppm toluene, by weight of the eszopiclone.

47. The eszopiclone of claim 46 having no more than about 600 ppm isobutyl acetate, no more than about 800 ppm isopropanol and/or no more than about 1350 ppm toluene.

48. The eszopiclone of claim 47 having no more than about 890 ppm toluene.

49. A method for preparing eszopiclone free base having no more than about 5000 ppm of isobutyl acetate, isopropanol or ethanol, and/or no more than about 1400 ppm of toluene as residual solvent(s) by weight of the eszopiclone, comprising crystallizing eszopiclone from a solvent selected from the group consisting of toluene, isobutyl acetate, isopropanol:water in a volume ratio of about 3:1 to about 10:1, ethanol:water in a volume ratio of about 1:1 to about 95:5, and mixtures thereof.

50. The method of claim 49, further comprising heating the mixture of the starting eszopiclone and the solvent.

51. The method of claim 50, wherein the heating is to reflux.

52. The method of claim 50, wherein the heating is to a temperature of about 30° C. to about 90° C.

53. The method of claim 52, wherein the heating is to a temperature of about 60° C. to about 80° C.

54. The method of claim 49, wherein the eszopiclone product is precipitated by cooling the mixture of the starting eszopiclone and the solvent.

55. The method of claim 54, wherein the cooling is to a temperature less than room temperature.

56. The method of claim 49, wherein the solvent is isopropanol:water in a volume ratio of about 7:1 to about 10:1.

57. The method of claim 49, wherein the solvent is ethanol:water in a volume ratio of about 3:1 to about 10:1.

58. The method of claim 49, wherein the solvent is ethanol:water in a volume ratio of about 95:5.

59. The method of claim 49, wherein the obtained eszopiclone has a chemical purity of more than about 97%.

60. The method of claim 49, wherein the obtained eszopiclone has an optical purity of at least about 97%.

61. The method of claim 49, wherein the obtained eszopiclone is eszopiclone Form A.
